(19) United States
(12) Patent Application Publication
(10) Pub. No.: US 2005/0148577 A1
(43) Pub. Date: Jul. 7, 2005

(54) 1-oxa-dibenzoazulenes as inhibitors of tumor necrosis factor production and intermediates for the preparation thereof

(75) Inventors: Mladen Mercep, Zagreb (HR); Milan Mesic, Zagreb (HR); Dijana Pesic, Sibenik (HR)

Correspondence Address: DARBY & DARBY P.C., P. O. BOX 5257, NEW YORK, NY, 10150-5257, US

(73) Assignee: Pliva-Istrazivacki Institut d.o.o., Zagreb (HR)

(21) Appl. No.: 10/995,954

(22) Filed: Nov. 22, 2004

Related U.S. Application Data
(63) Continuation-in-part of application No. PCT/HR03/00024, filed on May 20, 2003.

(30) Foreign Application Priority Data
May 21, 2002 (HR) P20020441A

(51) Int. Cl. A61K 31/55; A61K 31/343; A61K 31/38; C07D 495/02; C07D 493/02

(52) U.S. Cl. 514/215; 514/450; 514/431; 540/576; 549/12; 549/354

(57) ABSTRACT

The present invention relates to 1-oxa-dibenzoazulene derivatives, to their pharmacologically acceptable salts and solvates, to processes and intermediates for the preparation thereof as well as to their antiinflammatory effects, especially to the inhibition of tumour necrosis factor-α (TNF-α) production and the inhibition of interleukin-1 (IL-1) production as well as to their analgetic action.

### TECHNICAL FIELD

The present invention relates to 1-oxa-dibenzoazulene derivatives, to their pharmacologically acceptable salts and solvates, to processes and intermediates for the preparation thereof as well as to their antiinflammatory effects, especially to the inhibition of tumour necrosis factor-α (TNF-α) production and the inhibition of interleukin-1 (IL-1) production as well as to their analgetic action.

### PRIOR ART

There exist numerous literature data relating to various dibenzoazulenes of furan class and to the preparation thereof. It has been known that some tetracyclic tetrahydrofuran derivatives show antipsychotic, cardiovascular and gastrokinetic actions (WO 97/38991 and WO 99/19317). Described is also the preparation of 2-oxa-dibenzoazulene derivatives (U.S. Pat. No. 3,894,032; U.S. Pat. No. 3,974,285 and U.S. Pat. No. 4,044,143) and 2-oxa-8-thia-dibenzoazulenes (Tochtermann W, Chem. Ber., 1968, 101: 3122-3137; McHugh K B et al., J. Heterocycl. Chem., 1990, 27: 1839-42).

Likewise, there are known 1-thia-dibenzoazulene derivatives with aminoalkyloxy substituents on the thiophene ring showing antiimmflamatory action (WO 01/87890).

According to available literature data there are known 1-oxa-dibenzoazulene derivatives having phenyl, substituted phenyl (Becker H D et al., Tetrahedron Lett., 1985, 26: 1589-1592) or naphtyl (Mori Y et al., J. Chem. Soc., Perkin Trans. 2, 1996, 1: 113-119) in 2-position, whereas 1-oxa-dibenzoazulene derivatives of the present invention and especially those having aminoalkyloxy substituents on the furan ring have hitherto been neither prepared nor described. It has not been known either that such compounds would show antiimmflamatory (inhibitors of TNF-α secretion, inhibitors of IL-1 secretion) or analgetic action, which is also an object of the present invention.

In 1975 TNF-α was defined as a serum factor induced by endotoxin and causing tumour necrosis in vitro and in vivo (Carswell E A et al., Proc. Natl. Acad. Sci. U.S.A., 1975, 72: 3666-3670). Besides an antitumour action, TNF-α also possesses numerous other biological actions important in the homeostasis of an organism and in pathophysiological conditions. The main sources of TNF-α are monocytes-macrophages, T-lymphocytes and mastocytes.

The discovery that anti-TNF-α antibodies (cA2) have an action in treating patients with rheumatoid arthritis (RA) (Elliott M et al., Lancet, 1994, 344: 1105-1110) led to an increased interest in finding novel TNF-α inhibitors as possible potent drugs for RA. Rheumatoid arthritis is an autoimmune chronic inflammatory disease characterized by irreversible pathological changes in the joints. Besides in RA treatment, TNF-α antagonists may also be used in numerous pathological conditions and diseases such as spondylitis, osteoarthritis, gout and other arthritic conditions, sepsis, septic shock, toxic shock syndrom, atopic dermatitis, contact dermatitis, psoriasis, glomerulonephritis, lupus erythematosus, scleroderma, asthma, cachexia, chronic obstructive lung disease, congestive cardiac arrest, insulin resistance, lung fibrosis, multiple sclerosis, Crohn's disease, ulcerative colitis, viral infections and AIDS.

Some of the proofs indicating the biological importance of TNF-α were obtained by in vivo experiments in mice, in which mice gens for TNF-α or its receptor were inactivated. Such animals are resistant to collagen-induced arthritis (Mori L et al., J. Immunol., 1996, 157: 3178-3182) and to endotoxin-caused shock (Pfeffer K et al., Cell, 1993, 73: 457-467). In animal experiments where the TNF-α level was increased, a chronic inflammatory polyarthritis occured (Georgopoulos S et al., J. Inflamm., 1996, 46: 86-97; Keffer J et al., EMBO J., 1991, 10: 4025-4031) and its pathological picture was alleviated by inhibitors of TNF-α production. The treatment of such inflammatory and pathological conditions usually includes the application of non-steroid antiinflammatory drugs and, in more severe cases, gold salts, D-penicillinamine or methotrexate are administered. Said drugs act symptomatically, but they do not stop the pathological process. Novel approaches in the therapy of rheumatoid arthritis are based upon drugs such as tenidap, leflunomide, cyclosporin, FK-506 and upon biomolecules neutralizing the TNF-α action. At present there are commercially available etanercept (Enbrel, Immunex/Wyeth), a fusion protein of the soluble TNF receptor, and infliximab (Remicade, Centocor), a chimeric monoclonal human and mouse antibody. Besides in RA therapy, etanercept and infliximab are also registered for the therapy of Crohn's disease (Exp. Opin. Invest. Drugs, 2000, 9: 103).

In an optimal RA therapy, besides inhibition of TNF-α secretion, also the inhibition of IL-1 secretion is very important since IL-1 is an important cytokin in cell regulation and immunoregulation as well as in pathophysiological conditions such as inflammation (Dinarello C A et al., Rev. Infect. Disease, 1984, 6: 51). Well-known biological activities of IL-1 are: activation of T-cells, induction of elevated temperature, stimulation of secretion of prostaglandine or collagenase, chemotaxia of neutrophils and reduction of iron level in plasma (Dinarello C A, J. Clinical Immunology, 1985, 5: 287). Two receptors to which IL-1 may bind are well-known: IL-1RI and IL-1RII. IL-1RI transfers a signal intracellularly, whereas IL-1RII, though situated on the cell surface, does not transfer a signal inside the cell. Since IL1-RII binds IL-1 as well as IL1-RI, it may act as a negative regulator of IL-1 action. Besides this mechanism of signal transfer regulation, another natural antagonist of IL-1 receptor, IL-1ra, is present in cells. This protein binds to IL-1RI, but does not bring about a stimulation thereof. The potency of IL-1ra in stopping the signal transfer is not high and its concentration has to be 500 times higher than that of IL-1 in order to achieve a break in the signal transfer. Recombinant human IL-1ra (Amgen) was clinically tested (Bresnihan B et al., Arthrit. Rheum., 1996, 39: 73) and the obtained results indicated an improvement of the clinical picture in RA patients over a placebo. These results indicate the importance of the inhibition of IL-1 action in treating diseases such as RA where IL-1 production is disturbed. Since there exists a synergistic action of TNF-α and IL-1, dual TNF-α and IL-1 inhibitors may be used in treating conditions and diseases related to an enhanced secretion of TNF-α and IL-1.

### SOLUTION OF TECHNICAL PROBLEM

The present invention relates to compounds of 1-oxa-dibenzoazulenes of the formula I 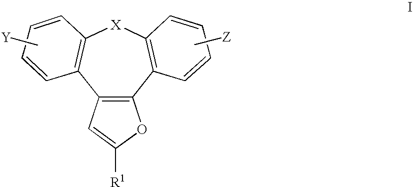
wherein
X may be CH₂ or a hetero atom such as O, S, S(═O), S(═O)₂, or NR^{a}, wherein R^{a} is hydrogen or a protecting group;
Y and Z independently from each other denote one or more identical or different substituents linked to any available carbon atom and may be halogen, C₁-C₄ alkyl, C₂-C₄ alkenyl, C₂-C₄ alkinyl, halo-C₁-C₄ alkyl, hydroxy, C₁-C₄ alkoxy, trifluoromethoxy, C₁-C₄ alkanoyl, amino, amino-C₁-C₄ alkyl, C₁-C₄ alkylamino, N-(C₁-C₄-alkyl)amino, N,N-di(C₁-C₄-alkyl)amino, thiol, C₁-C₄ alkylthio, sulfonyl, C₁-C₄ alkylsulfonyl, sulfinyl, C₁-C₄ alkylsulfinyl, carboxy, C₁-C₄ alkoxycarbonyl, cyano, nitro;
R¹ may be hydrogen, halogen, an optionally substituted C₁-C₇ alkyl or C₂-C₇ alkenyl, C₂-C₇ alkinyl, an optionally substituted heteroaryl or heterocycle, hydroxy, hydroxy-C₂-C₇ alkenyl, hydroxy-C₂-C₇ alkinyl, C₁-C₇alkoxy, thiol, thio-C₂-C₇ alkenyl, thio-C₂-C₇ alkinyl, C₁-C₇ alkylthio, amino, N-(C₁-C₇ alkyl)amino, N,N-di-(C₁-C₇ alkyl)amino, C₁-C₇ alkylamino, amino-C₂-C₇ alkenyl, amino-C₂-C₇ alkinyl, amino-C₁-C₇ alkoxy, C₁-C₇ alkanoyl, aroyl, oxo-C₁-C₇ alkyl, C₁-C₇ alkanoyloxy, carboxy, an optionally substituted C₁-C₇ alkyloxycarbonyl or aryloxycarbonyl, carbamoyl, N-(C₁-C₇-alkyl)carbamoyl, N,N-di(C₁-C₇-alkyl)carbamoyl, cyano, cyano-C₁-C₇ alkyl, sulfonyl, C₁-C₇ alkylsulfonyl, sulfinyl, C₁-C₇ alkylsulfinyl, nitro,
   or a substituent of the formula II 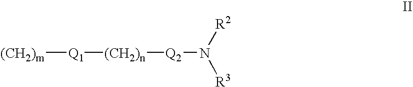
      wherein
R² and R³ simultaneously or independently from each other may be hydrogen, C₁-C₄ alkyl, aryl or together with N have the meaning of an optionally substituted heterocycle or heteroaryl;
m and n represent an integer from 0 to 3;
Q₁ and Q₂ represent, independently from each other, oxygen, sulfur or groups: 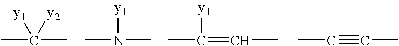
   wherein the substituents
y₁ and y₂ independently from each other may be hydrogen, halogen, an optionally substituted C₁-C₄ alkyl or aryl, hydroxy, C₁-C₄ alkoxy, C₁-C₄ alkanoyl, thiol, C₁-C₄ alkylthio, sulfonyl, C₁-C₄ alkylsulfonyl, sulfinyl, C₁-C₄ alkylsulfinyl, cyano, nitro or together form carbonyl or imino group;
   as well as to pharmacologically acceptable salts and solvates thereof.

The term “halo”, “hal” or “halogen” relates to a halogen atom which may be fluorine, chlorine, bromine or iodine.

The term “alkyl” relates to alkyl groups with the meaning of alkanes wherefrom radicals are derived, which radicals may be straight, branched or cyclic or a combination of straight and cyclic ones and branched and cyclic ones. The preferred straight or branched alkyls are e.g. methyl, ethyl, propyl, isopropyl, butyl, sec-butyl and tert-butyl. The preferred cyclic alkyls are e.g. cyclopentyl or cyclohexyl.

The term “haloalkyl” relates to alkyl groups which must be substituted with at least one halogen atom. The most frequent haloalkyls are e.g. chloromethyl, dichloromethyl, trifluoromethyl or 1,2-dichloropropyl.

The term “alkenyl” relates to alkenyl groups having the meaning of hydrocarbon radicals, which may be straight, branched or cyclic or are a combination of straight and cyclic ones or branched and cyclic ones, but having at least one carbon-carbon double bond. The most frequent alkenyls are ethenyl, propenyl, butenyl or cyclohexenyl.

The term “alkinyl” relates to alkinyl groups having the meaning of hydrocarbon radicals, which are straight or branched and contain at least one and at most two carbon-carbon triple bonds. The most frequent alkinyls are e.g. ethinyl, propinyl or butinyl.

The term “alkoxy” relates to straight or branched chains of alkoxy group. Examples of such groups are methoxy, propoxy, prop-2-oxy, butoxy, but-2-oxy or methylprop-2-oxy.

The term “aryl” relates to groups having the meaning of an aromatic ring, e.g. phenyl, as well as to fused aromatic rings. Aryl contains one ring with at least 6 carbon atoms or two rings with totally 10 carbon atoms and with alternating double (resonant) bonds between carbon atoms. The most freqently used aryls are e.g. phenyl or naphthyl. In general, aryl groups may be linked to the rest of the molecule by any available carbon atom via a direct bond or via a C₁-C₄ alkylene group such as methylene or ethylene.

The term “heteroaryl” relates to groups having the meaning of aromatic and partially aromatic groups of a monocyclic or bicyclic ring with 4 to 12 atoms, at least one of them being a hetero atom such as O, S or N, and the available nitrogen atom or carbon atom is the binding site of the group to the rest of the molecule either via a direct bond or via a C₁-C₄ alkylene group defined earlier. Examples of this type are thiophenyl, pyrrolyl, imidazolyl, pyridinyl, oxazolyl, thiazolyl, pyrazolyl, tetrazolyl, pirimidinyl, pyrazinyl, quinolinyl or triazinyl.

The term “heterocycle” relates to five-member or six-member, fully saturated or partly unsaturated heterocyclic groups containing at least one hetero atom such as O, S or N, and the available nitrogen atom or carbon atom is the binding site of the group to the rest of the molecule either via a direct bond or via a C₁-C₄ alkylene group defined earlier. The most frequent examples are morpholinyl, piperidyl, piperazinyl, pyrrolidinyl, pirazinyl or imidazolyl.

The term “alkanoyl” group relates to straight chains of acyl group such as formyl, acetyl or propanoyl.

The term “aroyl” group relates to aromatic acyl groups such as benzoyl.

The term “optionally substituted alkyl” relates to alkyl groups which may be optionally additionally substituted with one, two, three or more substituents. Such substituents may be halogen atom (preferably fluorine or chlorine), hydroxy, C₁-C₄ alkoxy (preferably methoxy or ethoxy), thiol, C₁-C₄ alkylthio (preferably methylthio or ethylthio), amino, N-(C₁-C₄)alkylamino (preferably N-methylamino or N-ethylamino), N,N-di(C₁-C₄-alkyl)-amino (preferably dimethylamino or diethylamino), sulfonyl, C₁-C₄ alkylsulfonyl (preferably methylsulfonyl or ethylsulfonyl), sulfinyl, C₁-C₄ alkylsulfinyl (preferably methylsulfinyl).

The term “optionally substituted alkenyl” relates to alkenyl groups optionally additionally substituted with one, two or three halogen atoms. Such substituents may be e.g. 2-chloroethenyl, 1,2-dichloroethenyl or 2-bromo-propene-1-yl.

The term “optionally substituted aryl, heteroaryl or heterocycle” relates to aryl, heteroaryl or heterocyclic groups which may be optionally additionally substituted with one or two substituents. The substituents may be halogen (preferably chlorine or fluorine), C₁-C₄ alkyl (preferably methyl, ethyl or isopropyl), cyano, nitro, hydroxy, C₁-C₄ alkoxy (preferably methoxy or ethoxy), thiol, C₁-C₄ alkylthio (preferably methylthio or ethylthio), amino, N-(C₁-C₄)alkylamino (preferably N-methylamino or N-ethylamino), N,N-di(C₁-C₄-alkyl)-amino (preferably N,N-dimethylamino or N,N-diethylamino), sulfonyl, C₁-C₄ alkylsulfonyl (preferably methylsulfonyl or ethylsulfonyl), sulfinyl, C₁-C₄ alkylsulfinyl (preferably methylsulfinyl).

When X has the meaning of NR^{a} and R^{a} has the meaning of a protecting group, then R^{a} relates to groups such as alkyl (preferably methyl or ethyl), alkanoyl (preferably acetyl), alkoxycarbonyl (preferably methoxycarbonyl or tert-butoxycarbonyl), arylmethoxycarbonyl (preferably benzyloxycarbonyl), aroyl (preferably benzoyl), arylalkyl (preferably benzyl), alkylsilyl (preferably trimethylsilyl) or alkylsilylalkoxyalkyl (preferably trimethylsilylethoxymethyl).

When R² and R³ together with N have the meaning of heteroaryl or heterocycle, this means that such heteroaryls or heterocycles have at least one carbon atom replaced by a nitrogen atom through which the groups are linked to the rest of the molecule. Examples of such groups are morpholine-4-yl, piperidine-1-yl, pyrrolidine-1-yl, imidazole-1-yl or piperazine-1-yl.

The term “pharmaceutically suitable salts” relates to salts of the compounds of the formula I and includes e.g. salts with C₁-C₄ alkylhalides (preferably methyl bromide, methyl chloride) (quaternary ammonium salts), with inorganic acids (hydrochloric, hydrobromic, phosphoric, metaphosphoric, nitric or sulfuric acids) or with organic acids (tartaric, acetic, citric, maleic, lactic, fumaric, benzoic, succinic, methane sulfonic orp-toluene sulfonic acids).

Some compounds of the formula I may form salts with organic or inorganic acids or bases and these are also included in the present invention.

Solvates (most frequently hydrates) which may be formed by the compounds of the formula I or salts thereof are also an object of the present invention.

Depending upon the nature of particular substituents, the compounds of the formula I may have geometric isomers and one or more chiral centres so that there can exist enantiomers or diastereoisomers. The present invention also relates to such isomers and mixtures thereof, including racemates.

The present invention also relates to all possible tautomeric forms of particular compounds of the formula I.

A further object of the present invention relates to the preparation of compounds of the formula I according to processes comprising:
a) a cyclisation of the compounds of the formula III: 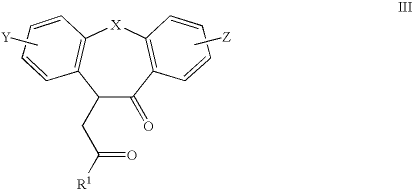
b) for the compounds of the formula I, wherein Q₁ has a meaning of —O—,
   a reaction of alcohols of the formula IV: 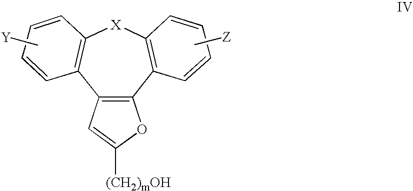
   with the compounds of the formula V: 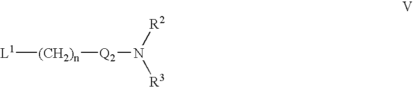
   wherein L¹ has the meaning of a leaving group;
c) for the compounds of the formula I, wherein Q₁ has a meaning of —O—, —NH—, —S— or —C≡C—,
   a reaction of the compounds of the formula IVa: 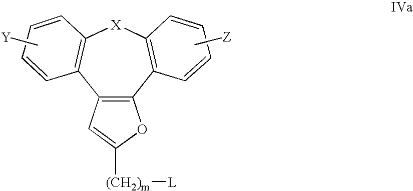
      wherein L has the meaning of a leaving group;
      with the compounds of the formula Va: 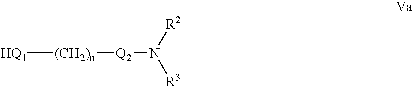
d) for the compounds of the formula I, wherein Q₁ has the meaning of —O—, —NH— or —S—,
   a reaction of the compunds of the formula IVb: 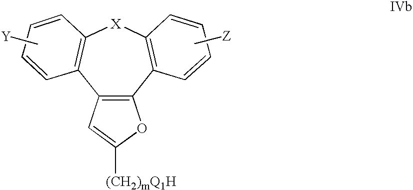
   with the compounds of the formula V, wherein L¹ has the meaning of a leaving group;
e) for the compounds of the formula I, wherein Q₁ has the meaning of —C═C—,
   a reaction of the compounds of the formula IVb, wherein Q₁ has the meaning of a carbonyl, with phospohorous ylides.

Preparation Methods:

a) Cyclization of the compounds of the formula III is carried out in toluene or benzene at boiling temperature during 1 to 5 hours in the presence of a catalytic amount of p-toluenesulfonic acid.

The starting reagents for the preparation of the compunds of the formula III are the compounds of the formula IIIa: 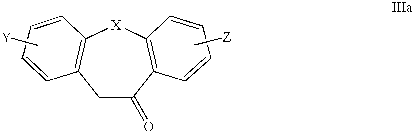
and the compounds of the formula IIIb: 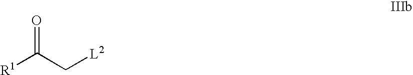
wherein L² has the meaning of a leaving group, which may be a halogen atom (most frequently bromine, iodine or chlorine). The reagents IIIa and IIIb are already known or are prepared according to methods disclosed for the preparation of analogous compounds.

The compounds of the formula III may be prepared in the presence of a strong base such as alkali hydrides (sodium hydride) or alkali amides (sodium amide) in a solvent such as dimethylformamide, dimethylsulfoxide or tetrahydrofuran at room temperature during 2 to 5 hours. The products may be isolated and purified by chromatography on a column, or may be, by means of cyclization, transferred into a corresponding furan derivative without isolation. A similar chemical sequence has already been described before [Iyer R N et al., Indian J. Chem., 1973, 11: 1260-1262].

b) The compounds of the formula I according to the present process may be prepared by reacting alcohols of the formula IV and compounds of the formula V, wherein L¹ has the meaning of a leaving group, which may be a halogen atom (most frequently bromine, iodine or chlorine) or a sulfonyloxy group (most frequently trifuloromethylsulfonyloxy or p-toluenesulfonyloxy). The condensation reaction may be carried out according to methods disclosed for the preparation of analogous compounds [Menozzi G., J. Heterocyclic Chem., 1997, 34: 963-968 or WO 01/87890]. The reaction is carried out at a temperature from 20° C. to 100° C. during 1 to 24 hours in a two-phase system (preferably with 50% NaOH/toluene) in the presence of a phase transfer catalyst (preferably benzyl triethyl ammonium chloride, benzyl triethyl ammonium bromide, cetyl trimethyl bromide). After the treatment of the reaction mixture, the products formed are isolated by recrystallization or chromatography on a silica gel column.

The starting compounds, alcohols of the formula IV, may be prepared from the compounds of the formula I, wherein R¹ has the meaning of a suitable functional group. So, e.g. the alcohols of the formula IV may be obtained by a reduction of an aldehyde, carboxyl or alkyloxycarbonyl group (e.g. methyloxycarbonyl or ethyloxycarbonyl) by use of metal hydrides such as lithium aluminum hydride or sodium borohydride. Further, the alcohols of the formula IV may be prepared by hydrolysis of the appropriate esters (in alkaline or acidic mediums).

The starting compounds of the formula V are already known or are prepared according to methods disclosed for the preparation of analogous compounds.

c) The compounds of the formula I according to the present process may be prepared by reacting compounds of the formula IVa, wherein L has the meaning of a leaving group defined earlier for L¹, and compounds of the formula Va, wherein Q₁ has the meaning of oxygen, nitrogen, sulfur or —C≡C—. The most suitable condensation reactions are reactions of nucleophilic substitution on a saturated carbon atom as disclosed in the literature.

The starting compounds of the formula IVa (most frequently halides) may be obtained by halogenation (e.g. bromination or chlorination) of compounds of the formula IV with the usual halogenating agents (hydrobromic acid, PBr₃, SOCl₂ or PCl₅) by processes as disclosed in the literature. The obtained compounds may be isolated or may be used without isolation as suitable intermediates for the preparation of the compounds of the formula I.

The starting compounds of the formula Va are already known or are prepared according to methods disclosed for the preparation of analogous compounds.

d) The compounds of the formula I, wherein Q₁ has the meaning of a hetero atom —O—, —NH— or —S—, may be prepared by the condensation of the compounds of the formula IVb and of compounds of the formula V, wherein L¹ has the meaning of a leaving group as defined earlier. The reaction may be carried out at reaction conditions disclosed in the method b) or at conditions of the nucleophilic substitution reactions disclosed in the literature. The starting alcohols, amines and thiols may be obtained by a reaction of water, ammonia or hydrogen sulfide with compounds IVa according to processes disclosed in the literature.

e) The alcohols of the structure IV may be oxidized to corresponding compounds of the formula IVb, wherein Q₁ has the meaning of carbonyl, which may further, by reaction with corresponding ylide reagents, result in a prolongation of the chain and in the formation of an alkenyl substituent with carbonyl or ester groups as disclosed in HR patent application No. 20000310.

Besides the above-mentioned reactions, the compounds of the formula I may be prepared by transforming other compounds of the formula I and it is to be understood that the present invention also comprises such compounds and processes. A special example of a change of a functional group is the reaction of the aldehyde group with chosen phosphorous ylides resulting in a prolongation of the chain and the formation of an alkenyl substituent with carbonyl or ester groups as disclosed in HR patent application No. 20000310. These reactions are carried out in solvents such as benzene, toluene or hexane at an elevated temperature (most frequently at boiling temperature).

By reacting the compounds of the formula IVa with 1-alkyne in an alkaline medium (such as sodium amide in ammonia), compounds of the formula I, wherein Q₁ is —C≡C—, are obtained. The reaction conditions of this process are disclosed in the literature. At similar reaction conditions (nucleophilic substitution) various ether, thioether or amine derivatives may be prepared.

The formylation of the compounds of the formula I by processes such as e.g. Vilsmeier acylation or reaction of n-BuLi and dimethylformamide is a further general example of a transformation. The reaction conditions of these processes are well-known in the literature.

By hydrolysis of the compounds of the formula I having nitrile, amide or ester groups, there may be prepared compounds with a carboxyl group, which are suitable intermediates for the preparation of other compounds with novel functional groups such as e.g. esters, amides, halides, anhydrides, alcohols or amines.

Oxidation or reduction reactions are a further possibility of the change of substituents in the compounds of the formula I. The most frequently used oxidation agents are peroxides (hydrogen peroxide, m-chloroperbenzoic acid or benzoyl peroxide) or permanganate, chromate or perchlorate ions. Thus e.g. by the oxidation of an alcohol group by pyridinyl dichromate or pyridinyl chlorochromate, an aldehyde group is formed, which may be converted to a carboxyl group by further oxidation. By oxidation of the compounds of the formula I, wherein R¹ has the meaning of alkyl, with lead tetraacetate in acetic acid or with N-bromosuccinimide using a catalytic amount of benzoyl peroxide, a corresponding carbonyl derivative is obtained.

By a selective oxidation of alkylthio group, alkylsulfinyl or alkylsulfonyl groups may be prepared.

By the reduction of the compounds with a nitro group, the preparation of amino compounds is made possible. The reaction is carried out under usual conditions of catalytic hydrogenation or electrochemically. By catalytic hydrogenation using palladium on carbon, alkenyl substituents may be converted to alkyl ones or the nitrile group can be converted to aminoalkyl.

Various substituents of aromatic structure in the compounds of the formula I may be introduced by standard substitution reactions or by usual changes of individual functional groups. Examples of such reactions are aromatic substitutions, alkylations, halogenation, hydroxylation as well as oxidation or reduction of substituents. Reagents and reaction conditions are known from the literature. Thus e.g. by aromatic substitution a nitro group is introduced in the presence of concentrated nitric acid and sulfuric acid. By using acyl halides or alkyl halides, the introduction of an acyl group or an alkyl group is made possible. The reaction is carried out in the presence of Lewis acids such as aluminum- or iron-trichloride in conditions of Friedel-Crafts reaction. By the reduction of the nitro group, an amino group is obtained, which is by a diazotizing reaction converted to a suitable starting group, which may be replaced with one of the following groups: H, CN, OH, Hal.

In order to prevent undesired interaction in chemical reactions, it is often necessary to protect certain groups such as e.g. hydroxy, amino, thio or carboxy. For this purpose a great number of protecting groups may be used [Green T W, Wuts P G H, Protective Groups in Organic Synthesis, John Wiley and Sons, 1999] and the choice, use and elimination thereof are conventional methods in chemical synthesis.

A convenient protection for amino or alkylamino groups are groups such as e.g. alkanoyl (acetyl), alkoxycarbonyl (methoxycarbonyl, ethoxycarbonyl or tert-butoxycarbonyl); arylmethoxycarbonyl (benzyloxycarbonyl), aroyl (benzoyl) or alkylsilyl (trimethylsilyl or trimethylsilylethoxymethyl) groups. The conditions of removing a protecting group depend upon the choice and the characteristics of this group. Thus e.g. acyl groups such as alkanoyl, alkoxycarbonyl or aroyl may be eliminated by hydrolysis in the presence of a base (sodium hydroxide or potassium hydroxide), tert-butoxycarbonyl or alkylsilyl (trimethylsilyl) may be eliminated by treatment with a suitable acid (hydrochloric, sulfuric, phosphoric or trifluoroacetic acid), whereas arylmethoxycarbonyl group (benzyloxycarbonyl) may be eliminated by hydrogenation using a catalyst such as palladium on carbon.

Salts of the compounds of the formula I may be prepared by generally known processes such as e.g. by reacting the compounds of the formula I with a corresponding base or acid in an appropriate solvent or solvent mixture e.g. ethers (diethylether) or alcohols (ethanol, propanol or isopropanol).

Another object of the present invention concerns the use of the present compounds in the therapy of inflammatory diseases and conditions, especially of all diseases and conditions induced by excessive TNF-α and IL-1 secretion.

The inhibitors of production of cytokins or inflammation mediators, which are the object of the present invention, or pharmacologically acceptable salts thereof may be used in the production of drugs for the treatment and prophylaxis of any pathological condition or disease induced by excessive unregulated production of cytokins or inflammation mediators, which drugs should contain an effective dose of said inhibitors.

The present invention specifically relates to an effective dose of TNF-α inhibitor, which may be determined by usual methods.

Further, the present invention relates to a pharmaceutical formulation containing an effective non-toxic dosis of the present compounds as well as pharmaceutically acceptable carriers or solvents.

The preparation of pharmaceutical formulations may include blending, granulating, tabletting and dissolving ingredients. Chemical carriers may be solid or liquid. Solid carriers may be lactose, sucrose, talcum, gelatine, agar, pectin, magnesium stearate, fatty acids etc. Liquid carriers may be syrups, oils such as olive oil, sunflower oil or soya bean oil, water etc. Similarly, the carrier may also contain a component for a sustained release of the active component such as e.g. glyceryl monostearate or glyceryl distearate. Various forms of pharmaceutical formulations may be used. Thus, if a solid carrier is used, these forms may be tablets, hard gelatine capsules, powder or granules that may be administered in capsules perorally (per os). The amount of the solid carrier may vary, but it is mainly from 25 mg to 1 g. If a liquid carrier is used, the formulation would be in the form of a syrup, emulsion, soft gelatine capsules, sterile injectable liquids such as ampoules or non-aqueous liquid suspensions.

Compounds according to the present invention may be applied per os, parenterally, locally, intranasally, intrarectally and intravaginally. The parenteral route herein means intravenous, intramuscular and subcutaneous applications. Appropriate formulations of the present compounds may be used in the prophylaxis as well as in the treatment of inflammatory diseases and conditions induced by an excessive unregulated production of cytokins or inflammation mediators, primarily TNF-α. They comprise e.g. rheumatoid arthritis, rheumatoid spondylitis, osteoarthritis and other arthritic pathological conditions and diseases, eczemas, psoriasis and other inflammatory skin conditions, inflammatory eye diseases, Crohn's disease, ulcerative colitis and asthma.

The inhibitory action of the present compounds upon TNF-α and IL-1 secretion was determined by the following in vitro and in vivo experiments:

Determination of TNF-α and IL-1 Secretion in Human Peripheral Blood Mononuclear Cells In Vitro

Human peripheral blood mononuclear cells (PBMC) were prepared from heparinized whole blood after separating PBMC on Ficoll-Paque™Plus (Amersham-Pharmacia). To determine the TNF-α level, 3.5-5×10⁴ cells were cultivated in a total volume of 200 μl for 18 to 24 hours on microtitre plates with a flat bottom (96 wells, Falcon) in RPMI 1640 medium, into which there were added 10% FBS (Fetal Bovine Serum, Biowhittaker) previously inactivated at 56° C./30 min, 100 units/ml of penicillin, 100 mg/ml of streptomycin and 20 mM HEPES (GIBCO). The cells were incubated at 37° C. in an atmosphere with 5% CO₂ and 90% humidity. In a negative control the cells were cultivated only in the medium (NC), whereas in a positive control TNF-α secretion was triggered by adding 1 ng/ml of lipopolysaccharides (LPS, E. coli serotype 0111:B4, SIGMA) (PC). The effect of the tested substances upon TNF-α secretion was investigated after adding them into cultures of cells stimulated by LPS (TS). The TNF-α level in the cell supernatant was determined by ELISA procedure according to the suggestions of the producer (R&D Systems). The test sensitivity was <3 pg/ml TNF-α. The IL-1 level was determined in an assay under the same conditions and with the same number of cells and the same concentration of stimulus by ELISA procedure (R&D Systems). The percentage of inhibition of TNF-α or IL-1 production was calculated by the equation:
% inhibition=[1−(TS−NC)/(PC−NC)]*100.

The IC₅₀ value was defined as the substance concentration, at which 50% of TNF-α production were inhibited.

Compounds showing IC₅₀ with 20 [M or lower concentrations are active.

Determination of TNF-α and IL-1 Secretion in Mouse Peritoneal Macrophages In Vitro

In order to obtain peritoneal macrophages, Balb/C mouse strain males, age 8 to 12 weeks, were injected i.p. with 300 μg of zymosan (SIGMA) dissolved in a phosphate buffer (PBS) in a total volume of 0.1 ml/mouse. After 24 hours the mice were euthanized according to the Laboratory Animal Welfare Act. The peritoneal cavity was washed with a sterile physiological solution (5 ml). The obtained peritoneal macrophages were washed twice with a sterile physiological solution and, after the last centrifugation (350 g/10 min), resuspended in RPMI 1640, into which 10% of FBS were added. In order to determine TNF-α secretion, 5×10⁴ cells/well were cultivated in a total volume of 200 μl for 18 to 24 hours on microtitre plates with a flat bottom (96 wells, Falcon) in RPMI 1640 medium, into which 10% FBS (Fetal Bovine Serum, Biowhittaker) inactivated by heat, 100 units/ml of penicillin, 100 mg/ml of streptomycin, 20 mM HEPES and 50 μM 2-mercaptoethanol (all of GIBCO) were added. The cells were incubated at 37° C. in an atmosphere with 5% CO₂ and 90% humidity. In a negative control the cells were cultivated only in a medium (NC), whereas in a positive control the TNF-α secretion was triggered by adding 10 ng/ml of lipopolysaccharides (LPS, E. coli serotype 0111:B4, SIGMA) (PC). The effect of the substances upon the TNF-α secretion was investigated after adding them into cultures of cells stimulated with LPS (TS). The TNF-α and IL-1 levels in the cell supernatant were determined by ELISA procedure specific for TNF-α and IL-1 (R&D Systems, Biosource). The percentage of inhibition of TNF-α or IL-1 production was calculated by the equation:
% inhibition=[1−(TS−NC)/(PC−NC)]*100.

The IC₅₀ value was defined as the substance concentration, at which 50% of TNF-α production were inhibited.

Compounds showing IC₅₀ with 10 μM or lower concentrations are active.

In Vivo Model of LPS-Induced Excessive TNF-α or IL-1 Secretion in Mice

TNF-α or IL-1 secretion in mice was induced according to the already disclosed method (Badger A M et al., J. Pharmac. Env. Therap., 1996, 279: 1453-1461). Balb/C males, age 8 to 12 weeks, in groups of 6 to 10 animals were used in the test. The animals were treated p.o. either with a solvent only (in negative and in positive controls) or with solutions of substances 30 minutes prior to i.p. treatment with LPS (E. coli serotype 0111:B4, Sigma) in a dosis of 1-25 μg/animal. Two hours later the animals were euthanized by means of i.p. Roumpun (Bayer) and Ketanest (Parke-Davis) injection. A blood sample of each animal was taken into a Vacutainer tube (Becton Dickinson) and the plasma was separated according to the producer's instructions. The TNF-α level in the plasma was determined by ELISA procedure (Biosource, R&D Systems) according to the producer's instructions. The test sensitivity was <3 pg/ml TNF-α. The IL-1 level was determined by ELISA procedure (R&D Systems). The percentage of inhibition of TNF-α or IL-1 production was calculated by the equation:
% inhibition=[1−(TS−NC)/(PC−NC)]*100.

Active are the compounds showing 30% or more inhibition of TNF-α production at a dosis of 10 mg/kg.

Writhing Assay for Analgetic Activity

In this assay pain is induced by the injection of an irritant, most frequently acetic acid, into the peritoneal cavity of mice. Animals react with characteristic writhings, which has given the name of the assay (Collier H O J et al., Pharmac. Chemother., 1968, 32: 295-310; Fukawa K et al., J. Pharmacol. Meth., 1980, 4: 251-259; Schweizer A et al., Agents Actions, 1988, 23: 29-31). The assay is convenient for the determination of analgetic activity of compounds. Procedure: male Balb/C mice (Charles River, Italy), age 8 to 12 weeks, were used. A control group received methyl cellulose p.o. 30 minutes prior to i.p. application of acetic acid in a concentration of 0.6%, whereas test groups received standard (acetylsalicylic acid) or test substances in methyl cellulose p.o. 30 minutes prior to i.p. application of 0.6% acetic acid (volume 0.1 ml/10 g). The mice were placed individually under glass funnels and the number of writhings was registered for 20 minutes for each animal. The percentage of writhing inhibition was calculated according to the equation:
% inhibition=(mean value of number of writhings in the control group−number of writhings in the test group)/number of writhings in the control group*100.

Active are the compounds showing such analgetic activity as acetylsalicylic acid or better.

In Vivo Model of LPS-Induced Shock in Mice

Male Balb/C mice (Charles River, Italy), age 8 to 12 weks, were used. LPS isolated from Serratie marcessans (Sigma, L-6136) was diluted in sterile physiological solution. The first LPS injection was administered intradermally in a dosis of 4 μg/mouse. 18 to 24 hours later, LPS was administered i.v. in a dosis of 90-200 μg/mouse. A control group received two LPS injections as disclosed above. The test groups received substances p.o. half an hour prior to each LPS application. Survival after 24 hours was observed.

Active are the substances at which the survival at a dosis of 30 mg/kg was 40% or more.

Compounds from Examples 4 to 7 show activity in at least two investigated assays though these results only represent an illustration of the biological activity of the compounds and should not limit the invention in any way.

### PREPARATION METHODS WITH EXAMPLES

The present invention is illustrated by the following Examples which are in no way a limitation thereof.

### Example 1

### 2-Methyl-1,8-dioxa-dibenzo[e,h]azulene (4)

To a solution of a compound 1 (1.5 mmoles) in benzene (20 ml) a catalytic amount of p-toluenesulfonic acid (p-TsOH) was added and the reaction mixture was heated at boiling temperature for 2-3 hours. Then the solvent was evaporated under reduced pressure, the dry residue was dissolved in a mixture of dichloromethane and water and the product was extracted by dichloromethane. The combined organic extracts were washed with a saturated NaHCO₃ solution and, after drying over anhydrous Na₂SO₄, the solvent was evaporated under reduced pressure. The crude product was purified by chromatography on a silica gel column and an oily yellow product was isolated.

According to the above process, starting from the compound 2, 11-chloro-2-methyl-1,8-dioxa-dibenzo[e,h]azulene (5) was prepared.

### Example 2

### a) 1,8-Dioxa-dibenzo[e,h]azulene-2-carbaldehyde (6)

To a solution of the compound 4 (0.4 mmole) in tetrachloromethane (10 ml) N-bromo-succinimide (NBS, 0.6 mmole) and a catalytic amount of benzoyl peroxide were added. The reaction mixture was stirred under heating at boiling temperature for 1-3 hours and then cooled to room temperature, the formed precipitate was filtered off and the filtrate was evaporated under reduced pressure. The dry residue was dissolved in a mixture of ethyl acetate and water and the organic product was extracted by ethyl acetate. By purification of the crude product on a silica gel column an oily light-yellow product was obtained.

### b) 11-Chloro-1,8-dioxa-dibenzo[e,h]azulene-2-carbaldehyde (7)

To a solution of the compound 5 (3.9 mmoles) in acetic acid (10 ml) lead tetraacetate (14 mmoles) was added and the reaction mixture was heated at boiling temperature for 2-3 hours. Then the solvent was evaporated and the dry residue was dissolved in a mixture of ethyl acetate and water. The organic product was extracted by ethyl acetate. After drying the organic extracts over anhydrous sodium sulfate and evaporation of the solvent, the crude product was purified on a silicagel column and an oily product was isolated.

### Example 3

### (1,8-Dioxa-dibenzo[e,h]azulene-2-il)-methanol (8)

To a suspension of LiAlH₄ (90 mg) in diethyl ether (10 ml) an ether solution of the compound 6 (0.34 mmole in 10 ml) was added. The reaction mixture was stirred at room temperature for 1-2 hours. The excess was hydrogenated by addition of a small quantity of a mixture of diethyl ether and water and the formed white precipitate was filtered off and washed with diethyl ether. After drying over anhydrous Na₂SO₄, the filtrate was evaporated and the obtained oily product was used in further synthesis steps without additional purification.

According to the above process, by reacting the compound 7 with LiAlH₄ in diethyl ether, the alcohol 11-chloro-1,8-dioxa-dibenzo[e,h]azulene-2-il)-methanol (9) was prepared.

| | |
|---|---|
| | I |
| 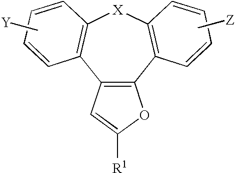 | |

| Comp. | X | Y | Z | R¹ | MS (m/z) | ¹H NMR (ppm, CDCl₃) |
|---|---|---|---|---|---|---|
| 4 | O | H | H | CH₃ | 303.1 | 2.44 (s, 3H); 6.39 (s, |
| | | | | | [M + | 1H); 7.13-7.58 (m, 8H) |
| | | | | | Na⁺ + | |
| | | | | | MeOH] | |
| 5 | O | H | 11-Cl | CH₃ | 337 | 2.43 (s, 3H); 6.39 (s, |
| | | | | | [M + | 1H); 7.15-7.36 (m, 6H); |
| | | | | | Na⁺ + | 7.52 (d, 1H) |
| | | | | | MeOH] | |
| 6 | O | H | H | CHO | 263.9 | 7.24-8.01 (m, 9H); |
| | | | | | [MH]⁺ | 9.76 (s, 1H) |
| 7 | O | H | 11-Cl | CHO | 297 | 7.22-7.45 (m, 6H); 7.57 |
| | | | | | [MH]⁺ | (s, 1H); 7.74 (d, 1H); |
| | | | | | | 9.77 (s, 1H) |
| 8 | O | H | H | CH₂OH | 265 | 1.9 (bs, 1H); 4.74 (s, |
| | | | | | [MH]⁺ | 2H); 6.72 (s, 1H); |
| | | | | | | 7.17-7.64 (m, 8H) |
| 9 | O | H | 11-Cl | CH₂OH | 301 | 2.24 (bs, 1H); 4.74 (s, |
| | | | | | [MH]⁺ | 2H); 6.7 (s, 1H); |
| | | | | | | 7.1-7.61 (m, 7H) |

### Example 4

### [3-(1,8-Dioxa-dibenzo[e,h]azulene-2-ylmethoxy)-propyl]-dimethyl-amine

(I; X=O, Y=Z=H, R¹=(CH₃)₂N(CH₂)₃OCH₂)

To a solution of 3-dimethylaminopropylchloride-hydrochloride (1.6 mmoles) in 50% sodium hydroxide (5 ml), benzyltriethylammnoium chloride (a catalytic amount) and a solution of the alcohol 8 (0.16 mmole) in toluene (10 ml) were added. The reaction mixture was heated under vigorous stirring at boiling temperature for 3-4 hours. Then it was cooled to room temperature, diluted with water and extracted with dichloromethane. The organic extract was washed with water, dried over anhydrous Na₂SO₄ and evaporated under reduced pressure. After purification of the evaporated residue by chromatography on a column, an oily product was isolated;

¹H NMR (ppm, CDCl₃): 2.04 (m, 2H); 2.53 (s, 6H); 2.76 (m, 12H); 3.69 (m, 2H); 4.59 (s, 2H); 6.75 (s, 1H); 7.19-7.65 (m, 8H);

MS (m/z): 350.1 [MH]⁺.

### Example 5

### [2-(11-Chloro-1,8-dioxa-dibenzo[e,h]azulene-2-ylmethoxy)-ethyl]-dimethyl-amine

(I; X=O, Y=H, Z=11-Cl, R¹=(CH₃)₂N(CH₂)₂OCH₂)

To a solution of 2-dimethylaminoethylchloride-hydrochloride (5.2 mmoles) in 50% sodium hydroxide (10 ml), benzyltriethylammnoium chloride (a catalytic amount) and a solution of the alcohol 9 (0.52 mmole) in toluene (10 ml) were added. The reaction mixture was heated under vigorous stirring at boiling temperature for 3-4 hours. Then it was cooled to room temperature, diluted with water and extracted with dichloromethane. The organic extract was washed with water, dried over anhydrous Na₂SO₄ and the solvent was evaporated under reduced pressure. After purification of the evaporated residue by chromatography on a column, an oily product was isolated;

MS (m/z): 370.4 [MH]⁺.

### Example 6

### [3-(1]-Chloro-1,8-dioxa-dibenzo[e,h]azulene-2-ylmethoxy)-propyl]-dimethyl-amine

(I; X=O, Y=H, Z=11-Cl, R¹=(CH₃)₂N(CH₂)₃OCH₂)

By a reaction of the alcohol 9 (0.52 mmoles) and 3-dimethylaminopropylchloride-hydrochloride (4.7 mmoles) according to the process described in Example 5, an oily product was obtained. MS (m/z): 384.4 [MH]⁺.

### Example 7

### 3-(11-Chloro-1,8-dioxa-dibenzo[e,h]azulene-2-ylmethoxy)-propylamine

(I; X=O, Y=H, Z=11-Cl, R¹=H₂N(CH₂)₃OCH₂)

By a reaction of the alcohol 9 (0.52 mmole) and 3-aminopropylchloride-hydrochloride (6.5 mmoles) according to the process described in Example 5, an oily product was obtained.

MS (m/z): 356.3 [MH]⁺.

Preparation of Starting Compounds

### 11-(2-Oxo-propyl)-11H-dibenzo[b,f]oxepin-10-one (1)

To a solution of 11H-dibenzo[b,f]oxepin-10-one (7.14 mmoles) in DMSO (15 ml), NaH (60% dispersion in a mineral oil, 0.5 g) was added. The reaction mixture was stirred at room temperature until the evolution of hydrogen had ceased (30-60 min), whereupon chlorine-acetone (25.3 mmoles) was added. After stirring for 3 hous at room temperature, a smaller quantity of water (in order to decompose the excess of hydride) was added to the reaction mixture and the organic product was extracted with dichloromethane. After drying on anhydrous sodium sulfate, the combined organic extracts were evaporated under reduced pressure. After purification of the crude product by chromatography on a silicagel column an oily light-yellow product was isolated. ¹H NMR (ppm, CDCl₃): 2.33 (s, 3H); 2.84-2.91 (dd, 1H); 3.64-3.80 (m, 1H); 4.93 (dd, 1H); 7.07-7.99 (m, 8H);

MS (m/z): 267 [MH]⁺.

According to the described process, starting from 8-chloro-11H-dibenzo[b,f]oxepin-10-one, there was prepared 8-chloro-11-(2-oxo-propyl)-11H-dibenzo[b,f]oxepin-10-one (2);

¹H NMR (ppm, CDCl₃): 2.36 (s, 3H); 2.85-2.92 (dd, 1H); 3.67-3.81 (m, 1H); 4.87-4.92 (m, 1H); 7.07-7.93 (m, 7H);

MS (m/z): 301 [MH]⁺;

and, starting from 11H-dibenzo[b,f]thiepin-10-one, there was prepared 11-(2-oxo-propyl)-11H-dibenzo[b,f]thiepin-10-one (3);

MS (m/z): 282.9 [MH]⁺.

1. A compound of the formula I 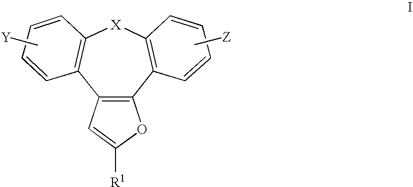
characterized in that
X may be CH₂ or a hetero atom such as O, S, S(═O), S(═OC)₂, or NR^{a}, wherein R^{a} is hydrogen or a protecting group;
Y and Z independently from each other denote one or more identical or different substituents linked to any available carbon atom, and may be hydrogen, halogen, C₁-C₄ alkyl, C₂-C₄ alkenyl, C₂-C₄ alkinyl, halo-C₁-C₄ alkyl, hydroxy, C₁-C₄ alkoxy, trifluoromethoxy, C₁-C₄ alkanoyl, amino, amino-C₁-C₄ alkyl, C₁-C₄ alkylamino, N-(C₁-C₄-alkyl)amino, N,N-di(C₁-C₄-alkyl)amino, thiol, C₁-C₄ alkylthio, sulfonyl, C₁-C₄ alkylsulfonyl, sulfinyl, C₁-C₄ alkylsulfinyl, carboxy, C₁-C₄ alkoxycarbonyl, cyano, nitro;
R¹ may be CHO or an optionally substituted C₁-C₇ alkyl;
as well as pharmacologically acceptable salts and solvates thereof.

2. A compound according to claim 1, characterized in that X represents O.

3. A compound according to claim 2, characterized in that Y represents H and Z represents H or Cl.

4. A compound according to claim 3, characterized in that R¹ represents CH₃, CHO, CH₂OH.

5. A compound of the formula I 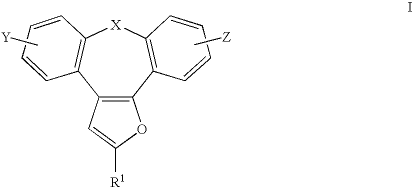
characterized in that
X may be CH₂ or a hetero atom such as O, S, S(═O), S(═O)₂, or NR^{a}, wherein R^{a} is hydrogen or a protecting group;
Y and Z independently from each other denote one or more identical or different substituents linked to any available carbon atom, and may be hydrogen, halogen, C₁-C₄ alkyl, C₂-C₄ alkenyl, C₂-C₄ alkinyl, halo-C₁-C₄ alkyl, hydroxy, C₁-C₄ alkoxy, trifluoromethoxy, C₁-C₄ alkanoyl, amino, amino-C₁-C₄ alkyl, C₁-C₄ alkylamino, IV-(C₁-C₄-alkyl)amino, N,N-di(C₁-C₄-alkyl)amino, thiol, C₁-C₄ alkylthio, sulfonyl, C₁-C₄ alkylsulfonyl, sulfinyl, C₁-C₄ alkylsulfinyl, carboxy, C₁-C₄ alkoxycarbonyl, cyano, nitro;
R¹ represents a substituent of the formula II 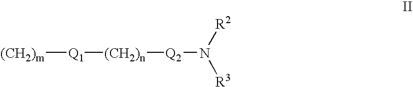
wherein
R² and R³ simultaneously or independently from each other may be hydrogen, C₁-C₄ alkyl, aryl or together with N have the meaning of an optionally substituted heterocycle or heteroaryl;
m and n represent an integer from 0 to 3;
Q₁ and Q₂ represent, independently from each other, oxygen, sulfur or groups: 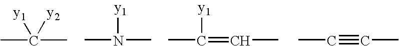
wherein the substituents
y₁ and y₂ independently from each other may be hydrogen, halogen, an optionally substituted C₁-C₄ alkyl or aryl, hydroxy, C₁-C₄ alkoxy, C₁-C₄ alkanoyl, thiol, C₁-C₄ alkylthio, sulfonyl, C₁-C₄ alkylsulfonyl, sulfinyl, C₁-C₄ alkylsulfinyl, cyano, nitro or together form carbonyl or imino group;
as well as pharmacologically acceptable salts and solvates thereof.

6. A compound according to claim 5, characterized in that X represents O.

7. A compound according to claim 6, characterized in that Y represents H and Z represents H or Cl.

8. A compound and a salt according to claim 7, characterized in that the symbol m has the meaning of 1, Q₁ represents O, n represents 1 or 2, Q₂ represents CH₂ and R² and R³ represent H or CH₃.

9. Selected compounds according to claim 4:
2-methyl-1,8-dioxa-dibenzo[e,h]azulene;
11-chloro-2-methyl-1,8-dioxa-dibenzo[e,h]azulene;
1,8-dioxa-dibenzo[e,h]azulene-2-carbaldehyde;
11-chloro-1,8-dioxa-dibenzo[e,h]azulene-2-carbaldehyde;
(1,8-dioxa-dibenzo[e,h]azulene-2-yl)-methanol;
(11-chloro-1,8-dioxa-dibenzo[e,h]azulene-2-yl)-methanol.

10. Selected compounds and salts according to claim 8:
[3-(1,8-dioxa-dibenzo[e,h]azulene-2-ylmethoxy)-propyl]-dimethyl-amine;
[2-(11-chloro-1,8-dioxa-dibenzo[e,h]azulene-2-ylmethoxy)-etil]-dimethyl-amine;
[3-(11-chloro-1,8-dioxa-dibenzo[e,h]azulene-2-ylmethoxy)-propyl]-dimethyl-amine;
3-(11-chloro-1,8-dioxa-dibenzo[e,h]azulene-2-ylmethoxy)-propylamine.

11. A process for the preparation of the compounds of the formula I 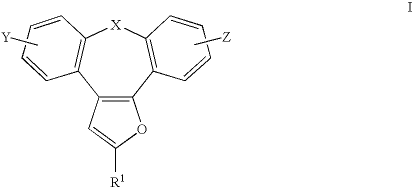
wherein
X may be CH₂ or a hetero atom such as O, S, S(═O), S(═O)₂, or NR^{a}, wherein R^{a} is hydrogen or a protecting group;
Y and Z independently from each other denote one or more identical or different substituents linked to any available carbon atom, and may be hydrogen, halogen, C₁-C₄ alkyl, C₂-C₄ alkenyl, C₂-C₄ alkinyl, halo-C₁-C₄ alkyl, hydroxy, C₁-C₄ alkoxy, trifluoromethoxy, C₁-C₄ alkanoyl, amino, amino-C₁-C₄ alkyl, C₁-C₄ alkylamino, N-(C₁-C₄-alkyl)amino, N,N-di(C₁-C₄-alkyl)amino, thiol, C₁-C₄ alkylthio, sulfonyl, C₁-C₄ alkylsulfonyl, sulfinyl, C₁-C₄ alkylsulfinyl, carboxy, C₁-C₄ alkoxycarbonyl, cyano, nitro;
R¹ may be CHO, an optionally substituted C₁-C₇ alkyl
or a substituent of the formula II 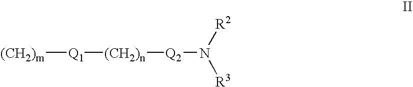
wherein
R² and R³ simultaneously or independently from each other may be hydrogen, C₁-C₄ alkyl, aryl or together with N have the meaning of an optionally substituted heterocycle or heteroaryl;
m and n represent an integer from 0 to 3;
Q₁ and Q₂ represent, independently from each other, oxygen, sulfur or groups: 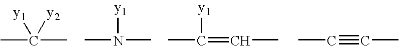
wherein the substituents
y₁ and y₂ independently from each other may be hydrogen, halogen, an optionally substituted C₁-C₄ alkyl or aryl, hydroxy, C₁-C₄ alkoxy, C₁-C₄ alkanoyl, thiol, C₁-C₄ alkylthio, sulfonyl, C₁-C₄ alkylsulfonyl, sulfinyl, C₁-C₄ alkylsulfinyl, cyano, nitro or together form carbonyl or imino group;
as well as of pharmacologically acceptable salts and solvates thereof.
characterized in that the process for the preparation comprises:
a) a cyclisation of the compounds of the formula III: 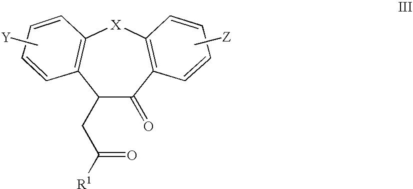
b) for the compounds of the formula I, wherein Q₁ has a meaning of —O—, a reaction of alcohols of the formula IV: 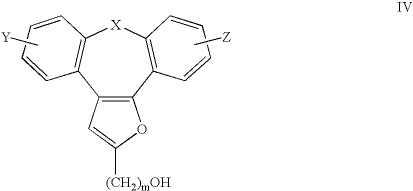
with the compounds of the formula V: 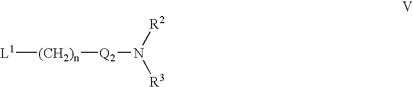
wherein L¹ has the meaning of a leaving group;
c) for the compounds of the formula I, wherein Q₁ has a meaning of —O—, —NH—, —S— or —C≡C—,
a reaction of the compounds of the formula IVa: 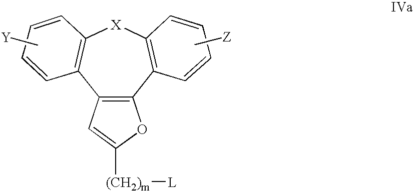
wherein L has the meaning of a leaving group;
with the compounds of the formula Va: 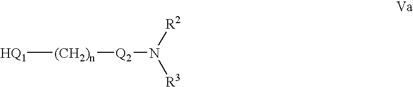
d) for the compounds of the formula I, wherein Q₁ has the meaning of —O—, —NH— or —S—,
a reaction of the compounds of the formula IVb: 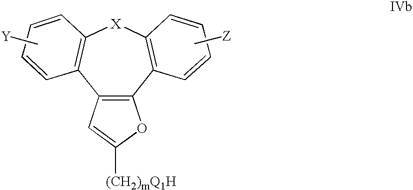
with the compounds of the formula V, wherein L¹ has the meaning of a leaving group;
e) for the compounds of the formula I, wherein Q₁ has the meaning of —C═C—, a reaction of the compounds of the formula IVb, wherein Q₁ has the meaning of a carbonyl, with phospohorous ylides.

12. Use of compounds of the formula I according to claim 7 as intermediates for the preparation of novel compounds of 1-oxa-dibenzoazulene class with antiimflammatory action.

13. Use of compounds of the formula I according to claim 5 as inhibitors of the production of cytokins or inflammation mediators for the treatment and prophylaxis of any pathological conditions or diseases induced by excessive unregulated production of cytokins or inflammation mediators by administering a nontoxic dosis of suitable pharmaceutical preparations perorally, parenterally or locally.
